(19) United States
(12) Patent Application Publication
(10) Pub. No.: US 2023/0347185 A1
(43) Pub. Date: Nov. 2, 2023

(54) PERSONA PROTECTIVE SUIT WITH CONTAMINANT PROTECTION

(71) Applicant: Frank Trung Leba, Portland, OR (US)

(72) Inventor: Frank Trung Leba, Portland, OR (US)

(21) Appl. No.: 17/753,125

(22) PCT Filed: Oct. 21, 2021
(86) PCT No.: PCT/US21/71979

§ 371 Date: Feb. 18, 2022

(51) Int. Cl. A62B 17/00; A62B 18/02; A62B 23/02; A62B 18/00; A61L 9/014; A61L 9/20; A41D 13/12

(52) U.S. Cl. CPC A62B 17/006; A62B 18/02; A62B 23/02; A62B 18/006; A61L 9/014; A61L 9/20; A41D 13/1218; A61L 2209/15; A61L 2209/12

(57) ABSTRACT

This present invention provides a system and device for protecting a wearer from hazardous materials and conditions while still allowing for freedom of movement.

### PRIORITY CLAIM

This application claims priority to and/or the benefit of U.S. provisional patent application Ser. No. 63/094,516 filed Oct. 21, 2020. The foregoing application is incorporated by reference in its entirety as if fully set forth herein.

### FIELD OF THE INVENTION

This invention relates generally to a system and device that functions as personal protective equipment.

### BACKGROUND OF THE INVENTION

Recent viral pandemics have increased public concern for public health and safety, especially in regard to mitigating potential viral or bacterial transmission of contaminants between infected persons and those providing care. In addition, there has been increased risk for providers of medical services who provide services to large numbers of persons and thus are at high risk of exposure. The cumbersome nature of existing methods of personal protective equipment (“PPE”) can make it difficult and physically taxing for providers to work over extended periods.

Increased awareness by the public of these costs creates the necessity for systems, methods, and devices, as disclosed herein, capable of assisting the public and private health workers by providing long term protection with reduced physical stress. Currently, conventional technologies and approaches to such problems are expensive, cumbersome, often require combining multiple protection methods, and/or are not always effective.

As such, this invention fulfills a vital need for highly safe personal protective equipment that is modular, easy to clean, easy to don and doff, meets and/or exceeds the requirements of the highest levels of protection demanded of PPE, breathable, reusable, and fluid-proof.

### BRIEF SUMMARY

The present invention is a protective suit that provides an isolated air supply that ensures that the air that the wearer breathes is filtered before it is inhaled and circulates the air out of the protective suit thereafter, ensuring that the wearer does not breath contaminated air without it being at least filtered beforehand. Current PPE models tend to lack protection, are overbuilt meaning require too many components, are difficult to use, and/or are difficult to maintain. The present invention solves these issues while providing a simple to operate and easy to maintain Level A PPE, granting a wearer complete protection against hazardous environments.

The present invention incorporates several components, including a flexible face shield, a filtration system, and a worn suit. In some embodiments the filtration system is a PTFE N99% BFE system that allows for optimal airflow while still protecting the wearer from N99% rated levels of particulates in the environment. The filtration system also incorporates an outflow that, in some embodiments, relies on a fan system to push air out of the suit and ensures that there is no air backflow, thus protecting the wearer further.

### BRIEF DESCRIPTION OF THE DRAWINGS

Embodiments of the present invention are described in detail below with reference to the following drawings:

FIG. 1 is a front view of a personal protective suit with an integrated filtration system in accordance with an embodiment of the invention;

FIG. 2 is a side view of a personal protective suit with an integrated filtration system in accordance with an embodiment of the invention;

FIG. 3 is a perspective view of a personal protective suit with an integrated filtration system in accordance with an embodiment of the invention;

FIG. 4 is a front view of a head cover of a personal protective suit with an integrated filtration system in accordance with an embodiment of the invention;

FIG. 5 is a side view of a head cover of a personal protective suit with an integrated filtration system in accordance with an embodiment of the invention;

FIG. 6 is a perspective view of a head cover of a personal protective suit with an integrated filtration system in accordance with an embodiment of the invention;

FIG. 7 is a rear perspective view of a head cover of a personal protective suit with an integrated filtration system in accordance with an embodiment of the invention;

FIG. 8 is a perspective view of the air ventilation system of an integrated filtration system in accordance with an embodiment of the invention;

FIG. 8 is a perspective view of the air ventilation system of an integrated filtration system in accordance with an embodiment of the invention;

FIG. 9 is a front view of the air ventilation system of an integrated filtration system in accordance with an embodiment of the invention;

FIG. 10 is a side view of the air ventilation system of an integrated filtration system in accordance with an embodiment of the invention;

FIG. 11 is a front view of a personal protective suit with an integrated filtration system and integrated gloves and boots in accordance with an embodiment of the invention;

FIG. 12 is a perspective view of the mask component of a personal protective suit with an integrated filtration system in accordance with an embodiment of the invention;

FIG. 13 is an exploded perspective view of the mask component of a personal protective suit with an integrated filtration system in accordance with an embodiment of the invention; and

FIG. 14 is a perspective, cross-section view of the air ventilation system of an integrated filtration system in accordance with an embodiment of the invention.

### DETAILED DESCRIPTION

This invention relates generally to a system and device comprising personal protective equipment. Specific details of certain embodiments of the invention are set forth in the following description and in the figures to provide a thorough understanding of such embodiments. The present invention may have additional embodiments, may be practiced without one or more of the details described for any particular described embodiment, or may have any detail described for one particular embodiment practiced with any other detail described for another embodiment.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

### DESCRIPTION OF INVENTION

This invention describes a protective garment capable of protecting a wearer against external threats in environments that may be highly toxic or otherwise hazardous and that possess a high risk of exposure to such. Specifically, this invention satisfies the requirements of the Centers for Disease Control (“CDC”) requirements for Level 4 biohazard. The invention is, however, modular and unburdensome such that it is easily suited for situations in which the need for protection is diminished, such as in CDC defined PPE Levels 1 through 3. As a result, the described invention allows for a user to freely perform most functions without hinderance from bulky or cumbersome equipment. The present invention also fulfills the requirements for PPE as defined by other organizations and government bodies.

The invention is comprised of a wearable suit featuring a few primary components; the primary body of the suit, head cover comprised of at least a faceplate assembly, and an air ventilation system. In some embodiments, additional components and functions may be added to the existing components. The body and at least a portion of the head cover of the suit, in many embodiments, will cover the wearer from the ankles to wrists to neck and be comprised of a fluid-proof material capable of resisting at least water. The suit and head cover may be comprised of a substance drawn from the list including, but not limited to, plastics, fabrics, rubbers, composites, hybrid materials, or a combination of the foregoing or other materials. Some embodiments of the suit may allow a fabric that is partially air permeable. Furthermore, in some embodiments the suit may be comprised of multiple layers of materials with each layer serving a different or collaborative purpose, such as, but not limited to, a layer of rubber to prevent fluid entry woven between layers of fabric to protect the rubber layer. The materials chosen for the components may also be selected for their ability to be cleaned and/or sterilized, as needed for a given embodiment.

The faceplate assembly, in many embodiments, is comprised of an air intake port, filter, and connection for the air ventilation system and couples with the suit to form a cavity of air over at least the wearer's nose and mouth. The air ventilation system, in many embodiments, is comprised of an air ventilation assembly configured with a means to draw air from the cavity of the faceplate and push it out, and may feature a sanitization system to sanitize the air before it exits the suit.

The body component of the suit may be further varied depending on the needs of the manufacturer or wearer, including by adding components such as integrated gloves or boots rather than ending at the wrist or ankle respectively. Some embodiments of the suit component may separate it into two or more pieces, such as, but not limited to, a top and bottom component that can be donned and doffed separately and may be secured together by an air-tight zipper or similar securing mechanism.

Some examples of the invention may be comprised of an air-flow assembly that is comprised of a fan system configured to pull air from the sealed portions of the suit, frequently the faceplate system but not exclusively, and push it out of the suit through an outlet. Some embodiments of the system may position the outlet at the top of the suit near the head, while others may position it in other locations or connect it to an external tube or ducting that assists in siphoning the air away. The air system may also, instead, rely on an external pump that draws air out of the system at a steady rate which is mitigated by the intake of air by the wearer, or may be configured to maintain a certain pressure with a weak vacuum to ensure that air leaves through the pump, duct, or tube system instead of through the filters. In such embodiments, the air ventilation assembly may be further comprised of an outlet that allows air to escape via a means determined by the manufacturer or wearer of the system, or the air ventilation system may simply be a valve or port that attaches to the faceplate assembly and connects to a separate hose.

Due to the difficulties of donning and doffing personal protective equipment, some embodiments of the invention are designed to be easy to don or doff. In some embodiments, the suit has a zipper integrated into the dorsal side such that it is zipped from the back, while in other embodiments a zipper or other airtight securing means may be located on the ventral or lateral sides of the suit. In other embodiments, the suit component may be donned by entering through a zipper that traces the medial facing portions of the legs where the inner side of the thigh, knee and calf would be located. Other mechanisms that secure the suit component can also be used, provided the seal of the faceplate remains intact, however non-fluid-proof mechanisms may invalidate the protection ratings of the invention. A beneficial feature of certain embodiments is that the air-tight cavity of air that is not contaminated is located between the seal, the wearer's face, and the faceplate, removing the issue of the wearer having to move around in a cumbersome inflated suit.

The present invention, in some embodiments, can be easily donned by having the wearer step into the suit component, placing their feet through the ankle cuffs and wrists through the wrist cuffs, then zipping the ventrally located zipper up to the wearer's neck. Following that, for at least one embodiment, the wearer places the head cover over their head such that the face shield is positioned over their head. In such examples, the wearer can then tighten draw strings, or other tightening mechanisms as appropriate, on the head cover to tighten the face shield against their hair line and jaw line, forming a cavity between their face and the faceplate of the head cover. In this example, the wearer need only activate the air ventilation system and they will be fully protected. The user can then doff the invention just as easily by reversing the process described above.

The present invention can be configured in a variety of different ways. For example, for some variations of the invention the suit component may have integrated gloves and/or boots, or they can be separate and secured via a cuff system. In some embodiments, the cuffs may have a self-sealing zipper mechanism or may utilize an elastic or similar means to ensure they close to form an air-tight seal around the wrists and ankles of the wearer, or to such other connection point. Alternatively, some embodiments may only extend to the knees and elbows, or to such other points as deemed appropriate by the wearer or manufacturer. Regardless of the embodiment, if the gloves and boots are a separate component, they can be connected anywhere as appropriate for the implementation provided the air-tight section formed by the faceplate is not eliminated.

The faceplate component is, in some embodiments, comprised of two primary parts; an outer housing comprising a substantially transparent plate with an integrated pocket system, and an insertable filtration pocket filter that fits into the integrated pocket system with the housing. For some examples of the faceplate, it may be comprised of a substance from the list including, but not limited to, glass, tempered glass, resins, plastics, polymers, polycarbonate, polyethylene terephthalate, amorphous copolyester, polyvinyl chloride, polypropylene, liquid silicone rubbers, cyclic olefin copolymers, polyethylene, styrene methyl methacrylate, styrene acrylonitrile resin, polystyrene, hybrid materials, combinations of the foregoing, or other at least materials. Non-translucent materials may also be employed in certain circumstances, depending on the needs of the manufacturer. For example, some manufacturers may inlay a polymer faceplate with a metallic mesh to provide strength.

The pocket-bladder system allows air to move through the filter system when the wearer breathes, and the type of filter inserted into the filter system can be varied depending on the needs of the wearer. In some embodiments, the pocket-bladder system may be an N95 filter, while in others it may be an N99, or other filtration mechanism as needed. In some embodiments, the pocket presses the filter against the air intake of the pocket, ensuring that any air being brought into the suit is brought in only through the air intake and thus must first pass through the filter. Some embodiments of the invention may feature a bladder-system wherein the filter is placed into the pocket of the faceplate assembly and is then zipped or secured through another means inside of the pocket. Some embodiments may secure the filter in the pocket through pressure with the air-tight seal, clips, or other mechanisms. In such embodiments, the pocket may have an external air intake port which allows air to enter into the pocket, flow through the filter, and enter into the cavity 114 between the faceplate and wearer's face through an internal air intake port when the wearer breaths. Some embodiments may feature the capability for the pocket to inflate to manage the atmospheric pressure in the faceplate when the wearer takes a breath.

The pocket-bladder system, in combination with the air ventilation system, can also assist in managing temperature for the wearer, which is a common problem for protective equipment. Wearers of PPE can often overheat and experience discomfort, or more severe side effects from prolonged use. The present invention, in some of its embodiments, is able to help manage temperature for the wearer through a few mechanisms. First, by sealing off the cavity alone, the rest of the body is free to be ventilated, in some embodiments, by materials that are breathable. Second, if present, a fan in the air ventilation system can help ensure cooling in the cavity. These features also help prevent humidity buildup and from there prevent fogging on the faceplate.

The faceplate, in some embodiments, is configured to form a seal with the suit or head cover, which may be sealed by a variety of means such as, but not limited to, pulling on a drawstring on the head cover or suit. Some examples of the faceplate will position the seal such that it substantially follows the hairline of the wearer to the ear, then the jawline to approximate a face shape of the wearer while creating an air-tight cavity between the seal, the wearer's face, and the faceplate itself. However, other variants may position such seals for the faceplate around the neck of the wearer, across their shoulders, over just the nose and mouth, or in a different configuration on the head. In such embodiments and other variations, the wearer breaths in, drawing air from outside through the filter and into the cavity. As the wearer breaths out, the air is taken up by the air ventilation system and then blown outside of the suit.

For some embodiments of the invention the head covering may be comprised of a hood component that covers the remainder of the head of the wearer and may extend down to the neck or shoulders. In other embodiments, the hood component may be integrated into the suit component, and may be removably couplable to the faceplate. The head cover component of the invention may, in some embodiments, cover the head and connect to the faceplate assembly, and then cover the wearer's neck and at least part of their shoulders in material which may be the same material that comprises the suit, or may be different if the needs of the embodiment require. In some embodiments the suit can be zipped over the head covering, while in others the head covering is configured to rest over the suit to form a seal.

To ensure a seal, many of the embodiments of the faceplate will have a drawstring, zipper, latch, zip-cord, or other mechanism to tighten the faceplate around some portion of the head of the wearer to form the cavity. Many examples of the faceplate securing mechanism will be adjustable such that they can fit a wide variety of wearers while still ensuring a fluid-tight seal so that the air in the cavity cannot leak out of the cavity without going through the sanitization system.

The faceplate component may be rigid or partially flexible; exact shape can vary to fit the faces of a variety of users. Depending on the method of manufacture, the faceplate can be adjusted for comfort and protection. The faceplate may be comprised of a variety of materials including, but not limited to, acrylics, plastics, polymers, glasses, or combinations thereof. The exact material composition of the faceplate can vary, and in many embodiments the faceplate will be at least partially translucent such that a wearer can see outside the suit. Advanced or combination materials may also be used, such as faceplates configured to transition to a tinted or polarized form when exposed to light. Other variations of the invention may utilize faceplates that are designed to block different wavelengths of light.

While some embodiments place the seal along the hair and jaw lines, the faceplate seal could be located at the neck, further forward, or elsewhere depending on the other components of the device and needs of the wearer. Some variations may alter the shape of the cavity or the position of the air ventilation system.

In some embodiments, the faceplate is further comprised of a filtration system, which may be comprised of a filter such as, but not limited to, an N95 rated filter configured to fit into an opening in the faceplate. In some embodiments, the faceplate may be comprised of a pocket in which to receive and hold the filter. The apertures in the faceplate for allowing air to enter the cavity of the faceplate may be located in the pocket, such that when a filter is placed into the pocket the filter covers the apertures, forcing any air that enters the cavity to pass through the filter.

The air ventilation system, in some embodiments, is comprised of an air-flow assembly and sanitation system. In some embodiments they may be separate components connected by a form of ducting, while in other embodiments they may be a singular component wherein air being pulled through the system by the air-flow assembly is passed through the sanitation system just before or just after sanitization.

For example, in some embodiments, the air ventilation system may be connected to an external air supply. In some embodiments, the body of the suit may be substantially in the form of a jumpsuit that encapsulates the wearer from ankles to wrists to neck, while in other embodiments the suit component may extend up the neck with a hood or similar component to substantially encapsulate the head of the wearer and connect to the faceplate assembly.

The sanitation system of the air ventilation system may be comprised of, in some embodiments, an ultraviolet-C(UVC) sanitation system wherein air leaving the system is passed through a UVC radiation source in order to sanitize it prior to leaving the suit's supply. In some embodiments, the air ventilation system 108 may further feature a filter to prevent unwanted particulates from leaving the cavity of the suit and to further ensure sanitation.

There are many mechanisms through which the air ventilation system can be powered; some embodiments may rely on an internal battery, while others may connect to an external power source. Some embodiments of the invention may combine an internal battery and external source, or may incorporate an alternative means of powering the air ventilation system or otherwise supporting a battery or external source, such as a solar panel located on the suit or faceplate, or through harnessing electricity from the wearer such as through their body heat.

In some embodiments, the air ventilation system may be configured to be controlled by a computer processor directly connected to the suit or connected by a network connection to the suit, allowing a wearer to alter parameters of the suit through a control unit such as an arm-mounted computer or through a device such as a mobile phone.

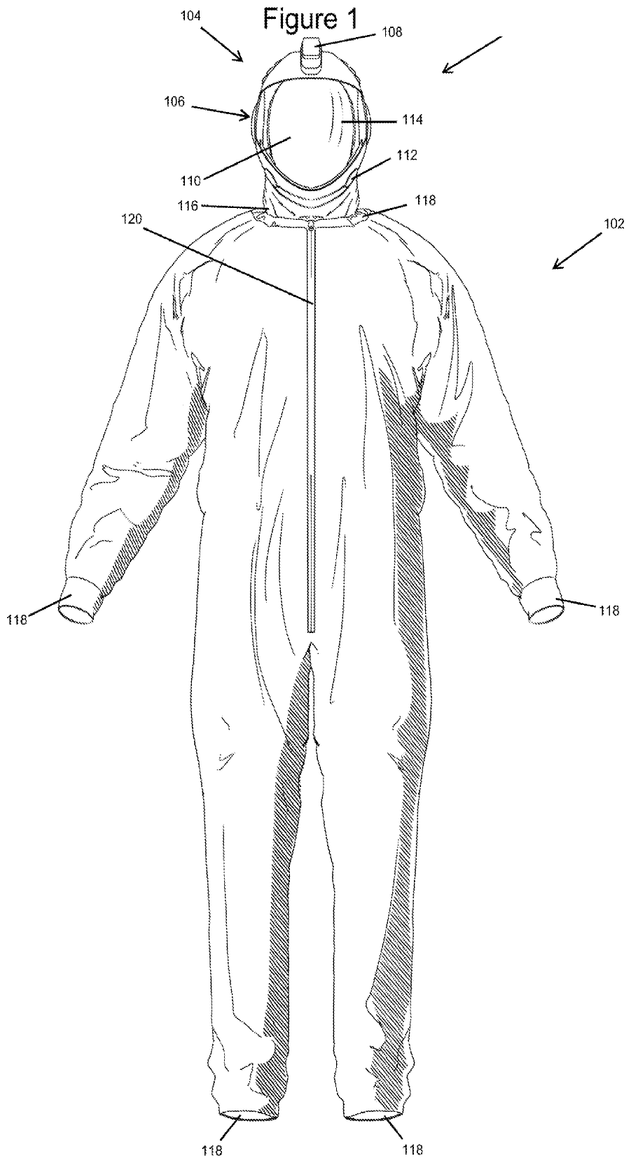

FIG. 1 is a front view of a personal protective suit with an integrated filtration system in accordance with an embodiment of the invention.

In some embodiments, the invention may be comprised of a personal protective suit 100 featuring three primary components; the body of the suit 102, head cover 104 featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket capable of receiving a filtering device wherein the pocket has at least one intake port 112; a hood 116 that substantially covers the head of the wearer and may also cover their neck and shoulders and extends to the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system 108. Depending on the embodiment, the air ventilation system 108 and pocket along with its intake port 112 may be positioned in a variety of locations; they are not limited to the respective top and bottom portions of the faceplate assembly 106. For example, some embodiments may place the air ventilation system 108 further back along the scalp of the wearer, or along the side of the wearer's head, while in some embodiments the pocket and intake port 112 may be positioned closer to the sides of the faceplate assembly 106, further up the faceplate assembly 106, on the back, or such other location provided that they allow, on their own or with other components, air to enter the cavity 114. In other embodiments, the air ventilation system 108 may be positioned somewhere else on the body of the wearer, such as for examples where tubing pumps air into the cavity or when the fluid-tight seal is positioned further back from the hairline and jawline of a wearer.

The invention, in some embodiments, may also be comprised of cuffs 118 positioned at the wrists, neck, and ankles of the suit 102 wherein such cuffs 118 are configured to tightly grip the body of the wearer such that fluids are at least partially prevented from entering the suit 102. In some embodiments, the suit has a zipper 120 integrated into the ventral side such that it is zipped from the front, while in other embodiments a zipper 120, zip-fastener, or other airtight securing means may be located on the dorsal or lateral sides of the suit.

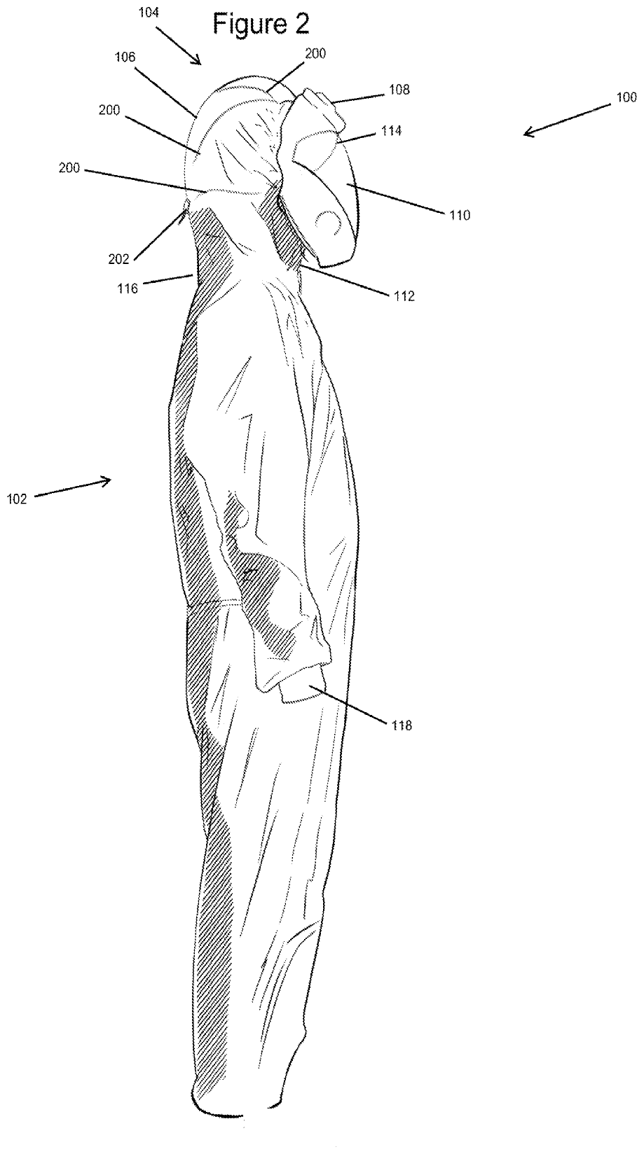

FIG. 2 is a side view of a personal protective suit with an integrated filtration system in accordance with an embodiment of the invention.

In some embodiments, the invention may be comprised of a personal protective suit 100 featuring three primary components; the body of the suit 102, head cover 104 featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket capable of receiving a filtering device wherein the pocket has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

The invention, in some embodiments, the suit 102 may also be comprised of cuffs 118 positioned at the wrists, neck, and ankles of the suit 102 wherein such cuffs 118 are configured to tightly grip the body of the wearer such that fluids are at least partially prevented from entering the suit 102. Some embodiments of the invention may feature a drawstring 200 or similar tightening mechanism coupled to the head cover 104 wherein the drawstring 200 has a pull 202 that can be drawn backward by a wearer.

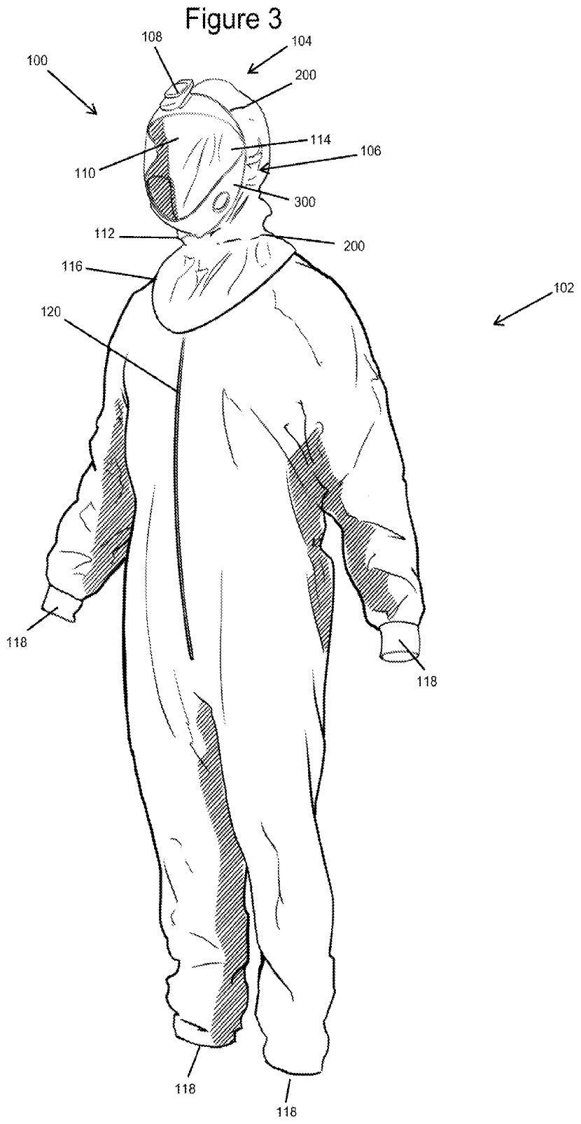

FIG. 3 is a perspective view of a personal protective suit with an integrated filtration system in accordance with an embodiment of the invention.

In some embodiments, the invention may be comprised of a personal protective suit 100 featuring three primary components; the body of the suit 102, head cover 104 featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

The invention, in some embodiments, may also be comprised of cuffs 118 positioned at the wrists, neck, and ankles of the suit 102 wherein such cuffs 118 are configured to tightly grip the body of the wearer such that fluids are at least partially prevented from entering the suit 102. In some embodiments, the suit has a zipper 120 integrated into the ventral side such that it is zipped from the front, while in other embodiments a zipper 120, zip-fastener, or other airtight securing means may be located on the dorsal or lateral sides of the suit.

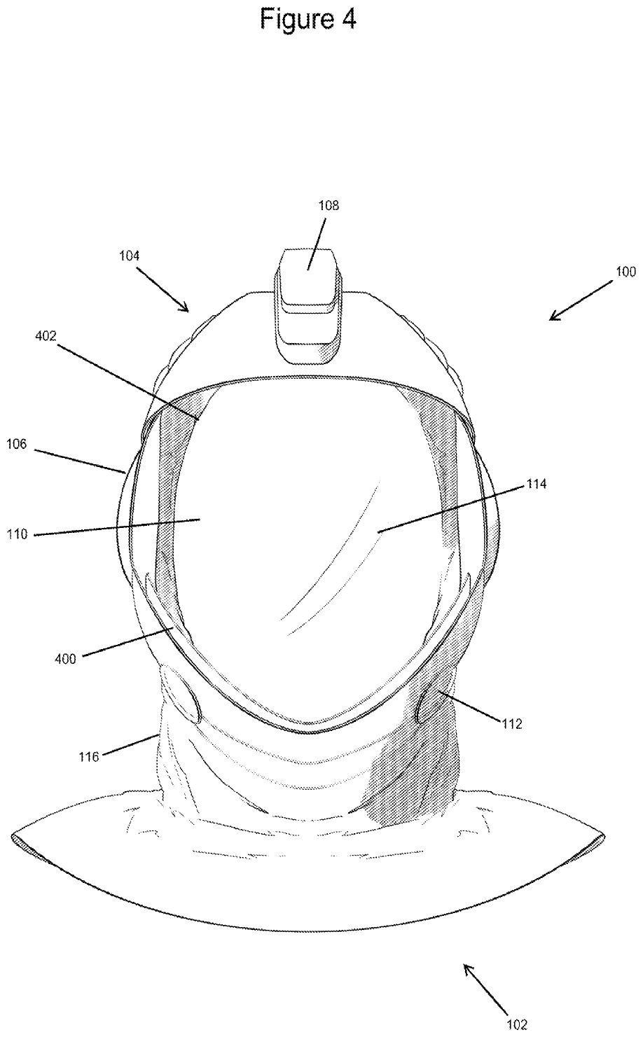

FIG. 4 is a front view of a head cover of a personal protective suit with an integrated filtration system in accordance with an embodiment of the invention.

In some embodiments, the invention may be comprised of a personal protective suit 100 featuring three primary components; the body of the suit, head cover 104 featuring a faceplate assembly 106, and an air ventilation system 108. In some embodiments, additional components and functions may be added to the existing components. The body of the suit, in many embodiments, will cover the wearer from the ankles to wrists to neck while the head cover 104 may cover the wearer's shoulders, neck, and head, and both may be comprised of a fluid-proof material capable of resisting at least water, but in many embodiments they will be configured to be impermeable to other fluids. Some embodiments of the suit and head cover 104 may allow a fabric that is partially air permeable. The faceplate assembly 106, in many embodiments, is comprised of a faceplate 110, an intake port 112, filter 400, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one intake port 112; a hood 116 that substantially covers the head of the wearer and may also cover their neck and shoulders and extends to the faceplate 110 and forms a fluid-tight seal 402 with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

In some embodiments the fluid-tight seal 402 with the faceplate 110 is located along the line formed by the hairline and jawline of the wearer such that only the wearer's face is in the cavity 114, while in others it may be positioned in other locations such as the neck, or further back along the head such that the ear is included in the cavity 114. The fluid-tight seal 402 may, in some embodiments, be formed of the back of the faceplate assembly 106 along with material from the hood 116 that is held or pressed against the face of the wearer by tension on a drawstring. In other embodiments, the pressure may be held by a clasp, clip, zipper, or other sealing mechanism.

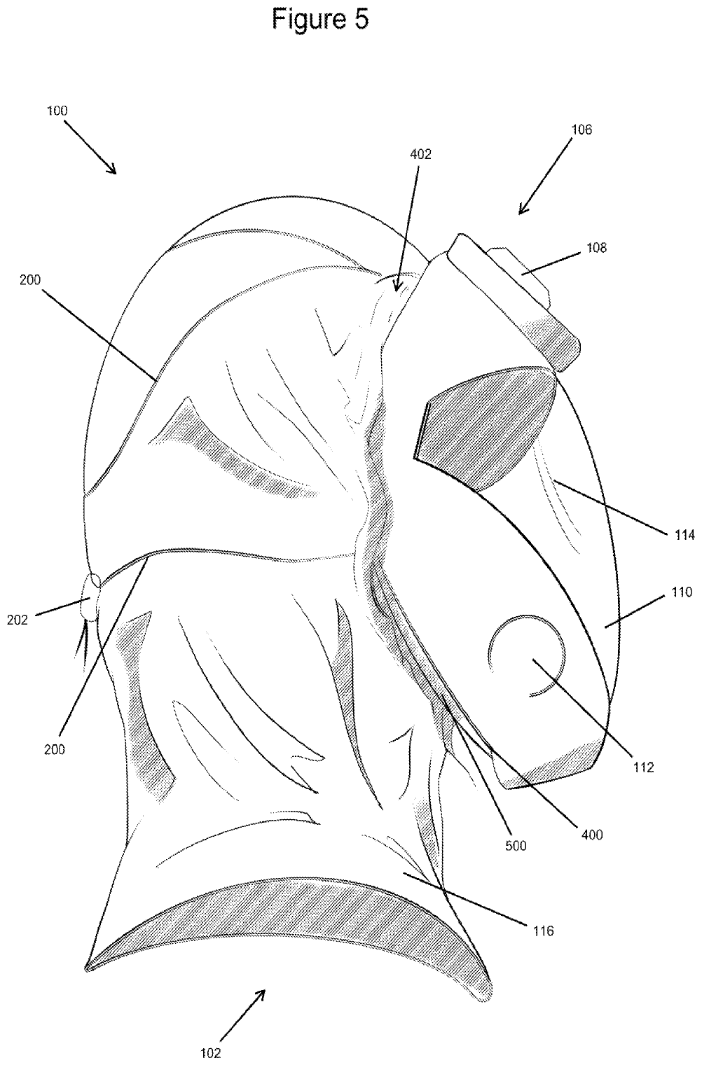

FIG. 5 is a side view of a head cover of a personal protective suit with an integrated filtration system in accordance with an embodiment of the invention.

In some embodiments, the invention may be comprised of a personal protective suit 100 featuring three primary components; the body of the suit, head cover 104 featuring a faceplate assembly 106, and an air ventilation system 108. In some embodiments, additional components and functions may be added to the existing components. The body of the suit, in many embodiments, will cover the wearer from the ankles to wrists to neck while the head cover 104 may cover the wearer's shoulders, neck, and head, and both may be comprised of a fluid-proof material capable of resisting at least water, but in many embodiments they will be configured to be impermeable to other fluids. Some embodiments of the suit and head cover 104 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket capable of receiving a filtering device wherein the pocket 400 has at least one intake port 112 and the filtering device is secured in the pocket by a zipper 500 which seals the filtering device into the pocket 400; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

Some embodiments of the invention may feature a drawstring 200 or similar tightening mechanism coupled to the head cover 104 wherein the drawstring 200 has a pull 202 that can be drawn backward by a wearer. In some embodiments, the suit has a zipper 120 integrated into the ventral side such that it is zipped from the front, while in other embodiments a zipper 120, zip-fastener, or other airtight securing means may be located on the dorsal or lateral sides of the suit.

In some embodiments the fluid-tight seal 402 with the faceplate 110 is located along the line formed by the hairline and jawline of the wearer such that only the wearer's face is in the cavity 114, while in others it may be positioned in other locations such as the neck, or further back along the head such that the ear is included in the cavity 114. The fluid-tight seal 402 may, in some embodiments, be formed of the back of the faceplate assembly 106 along with material from the hood 116 that is held or pressed against the face of the wearer by tension on a drawstring 200. In other embodiments, the pressure may be held by a clasp, clip, zipper, or other sealing mechanism.

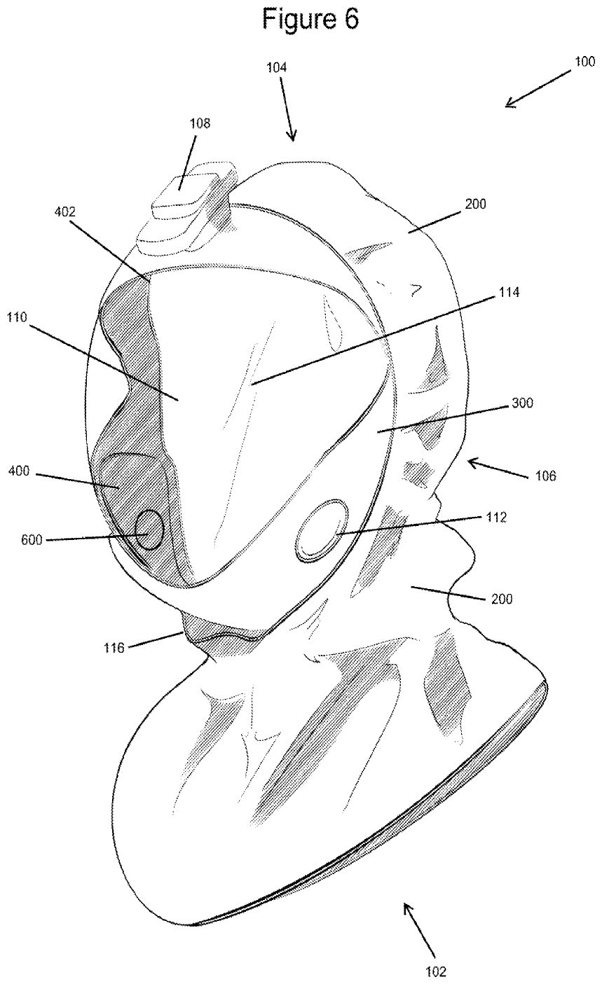

FIG. 6 is a perspective view of a head cover of a personal protective suit with an integrated filtration system in accordance with an embodiment of the invention.

In some embodiments, the invention may be comprised of a personal protective suit 100 featuring three primary components; the body of the suit, head cover 104 featuring a faceplate assembly, and an air ventilation system 108. In some embodiments, additional components and functions may be added to the existing components. The body of the suit, in many embodiments, will cover the wearer from the ankles to wrists to neck while the head cover 104 may cover the wearer's shoulders, neck, and head, and both may be comprised of a fluid-proof material capable of resisting at least water, but in many embodiments they will be configured to be impermeable to other fluids. Some embodiments of the suit and head cover 104 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one external intake port 112 and at least one internal intake port 600 capable of releasing air from the pocket 400 into the cavity 114; a hood 116 that substantially covers the head of the wearer and may also cover their neck and shoulders and extends to the faceplate 110 and forms a fluid-tight seal 402 with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system. In many embodiments, them will be two external intake ports 112 that correspondence with two internal intake ports 600 through which air is taken in, passed through the filter inside the pocket 400, and allowed into the cavity 114.

In some embodiments the fluid-tight seal 402 with the faceplate 110 is located along the line formed by the hairline and jawline of the wearer such that only the wearer's face is in the cavity 114, while in others it may be positioned in other locations such as the neck, or further back along the head such that the ear is included in the cavity 114. The fluid-tight seal 402 may, in some embodiments, be formed of the back of the faceplate assembly 106 along with material from the hood 116 that is held or pressed against the face of the wearer by tension on a drawstring 200. In other embodiments, the pressure may be held by a clasp, clip, zipper, or other sealing mechanism.

Some embodiments of the invention may feature a drawstring or similar tightening mechanism coupled to the head cover 104 wherein the drawstring has a pull 202 that can be drawn backward by a wearer.

The pocket 400 system allows air to move through the external intake port 112, internal intake port 600, and filter system when the wearer breathes, and the type of filter inserted into the pocket 400 system can be varied depending on the needs of the wearer. In some embodiments, the filter may be an N95 filter, while in others it may be an N99, or other filtration mechanism as needed. In some embodiments, the pocket 400 presses the filter against the external air intake 112 of the pocket 400, ensuring that any air being brought into the suit is brought in only through the air intake and thus must first pass through the filter. Some embodiments of the invention may configure the pocket 400 as a bladder-system wherein the filter is placed into the pocket 400 of the faceplate assembly 106 and is then zipped or secured through another means inside of the pocket 400, and the pocket 400 is able to expand and compress with atmospheric changes. Some embodiments may secure the filter in the pocket 400 through pressure with the fluid-tight seal 402, clips, or other mechanisms. In such embodiments, the pocket may have an external air intake port 112 which allows air to enter into the pocket, flow through the filter, and enter into the cavity 114 between the faceplate and wearer's face through an internal air intake port 600 when the wearer breaths. Some embodiments may feature the capability for the pocket 400 to inflate to manage the atmospheric pressure in the faceplate assembly 106 when the wearer takes a breath.

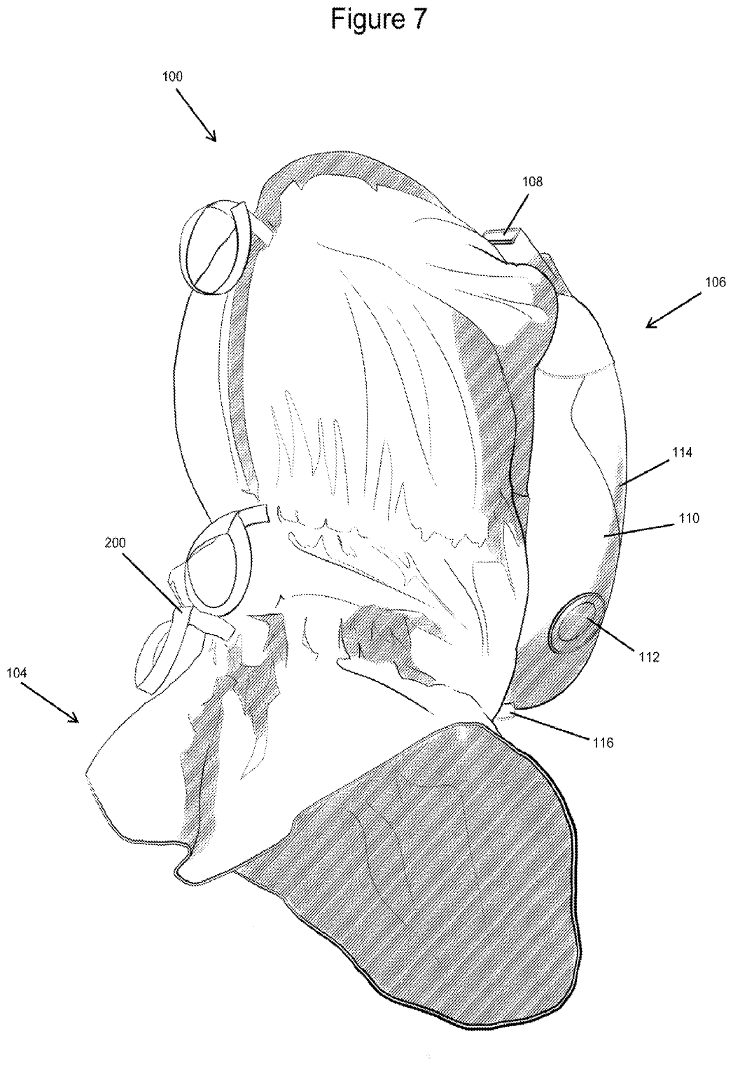

FIG. 7 is a rear perspective view of a head cover of a personal protective suit with an integrated filtration system in accordance with an embodiment of the invention;

In some embodiments, the invention may be comprised of a personal protective suit 100 featuring three primary components; the body of the suit, head cover 104 featuring a faceplate assembly 106, and an air ventilation system 108. In some embodiments, additional components and functions may be added to the existing components. The body of the suit, in many embodiments, will cover the wearer from the ankles to wrists to neck while the head cover 104 may cover the wearer's shoulders, neck, and head, and both may be comprised of a fluid-proof material capable of resisting at least water, but in many embodiments they will be configured to be impermeable to other fluids. Some embodiments of the suit and head cover 104 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

Some embodiments of the invention may feature a drawstring 200 or similar tightening mechanism coupled to the head cover 104 wherein the drawstring 200 has a pull that can be drawn backward by a wearer.

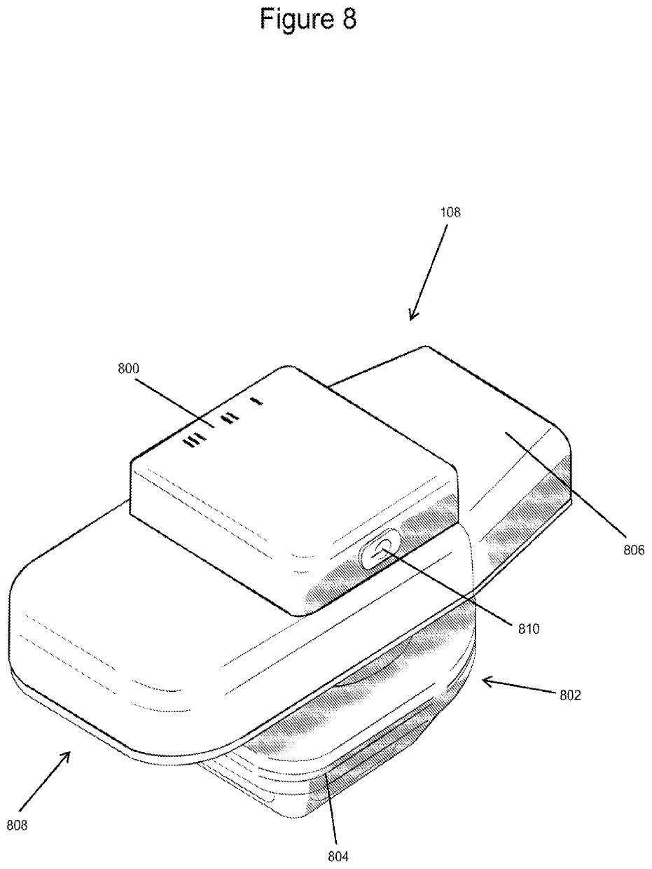

FIG. 8 is a perspective view of the air ventilation system of an integrated filtration system in accordance with an embodiment of the invention.

The air ventilation system 108, in some embodiments, is comprised of an air-flow assembly 800, a sanitation system 802, a power supply, a connection 804 to the faceplate, an exhaust port 806, and a casing 808. In some embodiments they may be separate components connected by a form of ducting, while in other embodiments they may be a singular component wherein air being pulled through the system by the air-flow assembly 800 is passed through the sanitation system 802 just before or just after it is taken into the fan. In some configurations, the air ventilation system 108 functions by operating a fan configured to push air out of the cavity of the faceplate, and may be further comprised of a door, hatch, or similar blocking mechanism capable of ensuring that air cannot enter the faceplate when the system is inoperative. Some configurations may involve the blocking mechanism being forced open by the increased air pressure that occurs in the cavity of the faceplate when the wearer exhales, in which case the fan further pushes air out of the exhaust port 806 while the door is opened. In other embodiments, the door or blocking mechanism is closed when the fan is turned off to ensure air cannot leak out of the suit, and the door or blocking mechanism then opens when the fan is turned on.

In at least one embodiment, the connection 804 of the air ventilation system 108 extends at least partially into the cavity of the faceplate wherein are located one or more air intake apertures which connect to the fan assembly, which in turn leads to the blocking mechanism, and through that mechanism is an exhaust port 806 where air is forced by the fan out of the cavity of the suit. The air ventilation system may have an integrated battery to power the fan and blocking mechanism. In some embodiments, the air ventilation system may have one or more fan speed settings.

For example, in some embodiments, the air ventilation system 108 may be connected to an external air supply that may connect at the exhaust port 806 or at the connection to the faceplate.

The sanitation system 802 of the air ventilation system 108 may be comprised of, in some embodiments, an ultraviolet-C(UVC) sanitation system wherein air leaving the system is passed through a UVC radiation source in order to sanitize it prior to leaving the suit's supply. In some embodiments, the air ventilation system 108 may further feature a filter to prevent unwanted particulates from leaving the cavity of the suit and to further ensure sanitation.

There are many mechanisms through which the air ventilation system 108 can be powered; some embodiments may rely on an internal battery, while others may connect to an external power source. Some embodiments of the invention may combine an internal battery and external source, or may incorporate an alternative means of powering the air ventilation system 108 or otherwise supporting a battery or external source, such as a solar panel located on the suit or faceplate, or through harnessing electricity from the wearer such as through their body heat.

In some embodiments, the air ventilation system 108 may be configured to be controlled by a computer processor directly connected to the suit or connected by a network connection to the suit, allowing a wearer to alter parameters of the suit through a control unit such as an arm-mounted computer or through a device such as a mobile phone. In some embodiments, the air ventilation system 108 may be controlled by a button 810, toggle, or other input located on the casing 808 which may control settings of the air ventilation system 108 or its activation or deactivation.

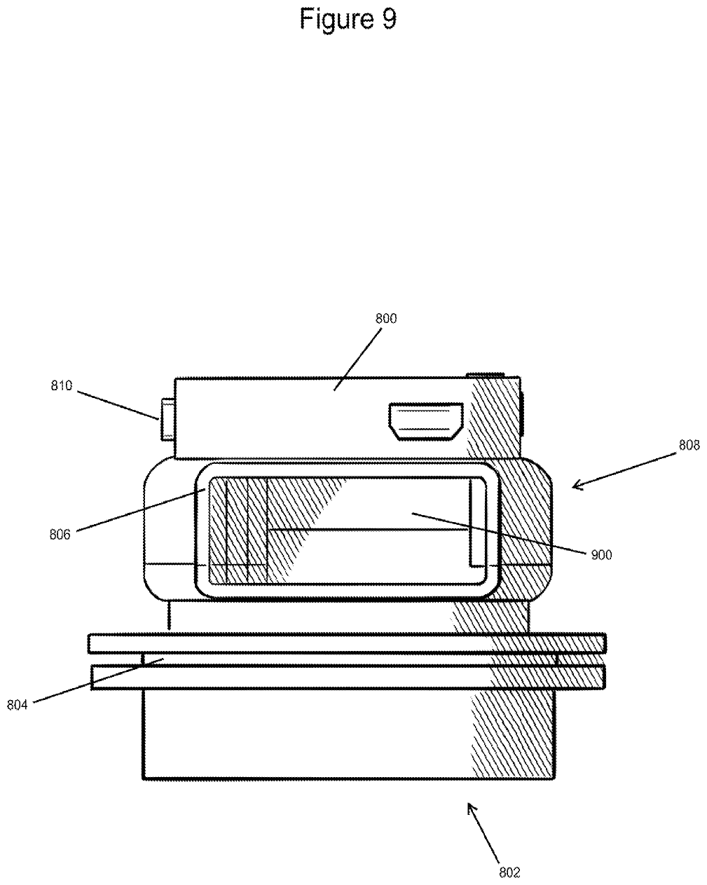

FIG. 9 is a front view of the air ventilation system of an integrated filtration system in accordance with an embodiment of the invention.

The air ventilation system 108, in some embodiments, is comprised of an air-flow assembly 800, a sanitation system 802, a power supply, a connection 804 to the faceplate, an exhaust port 806, and a casing 808. In some embodiments they may be separate components connected by a form of ducting, while in other embodiments they may be a singular component wherein air being pulled through the system by the air-flow assembly 800 is passed through the sanitation system 802 just before or just after it is taken into the fan. In some configurations, the air ventilation system 108 functions by operating a fan configured to push air out of the cavity of the faceplate, and may be further comprised of a door 900, hatch, or similar blocking mechanism capable of ensuring that air cannot enter the faceplate when the system is inoperative. Some configurations may involve the blocking mechanism being forced open by the increased air pressure that occurs in the cavity of the faceplate when the wearer exhales, in which case the fan further pushes air out of the exhaust port 806 while the door is opened. In other embodiments, the door or blocking mechanism is closed when the fan is turned off to ensure air cannot leak out of the suit, and the door or blocking mechanism then opens when the fan is turned on.

In at least one embodiment, the connection 804 of the air ventilation system 108 extends at least partially into the cavity of the faceplate wherein are located one or more air intake apertures which connect to the fan assembly, which in turn leads to the blocking mechanism, and through that mechanism is an exhaust port 806 where air is forced by the fan out of the cavity of the suit. The air ventilation system may have an integrated battery to power the fan and blocking mechanism. In some embodiments, the air ventilation system may have one or more fan speed settings.

For example, in some embodiments, the air ventilation system 108 may be connected to an external air supply that may connect at the exhaust port 806 or at the connection to the faceplate.

The sanitation system 802 of the air ventilation system 108 may be comprised of, in some embodiments, an ultraviolet-C(UVC) sanitation system wherein air leaving the system is passed through a UVC radiation source in order to sanitize it prior to leaving the suit's supply. In some embodiments, the air ventilation system 108 may further feature a filter to prevent unwanted particulates from leaving the cavity of the suit and to further ensure sanitation.

There are many mechanisms through which the air ventilation system 108 can be powered; some embodiments may rely on an internal battery, while others may connect to an external power source. Some embodiments of the invention may combine an internal battery and external source, or may incorporate an alternative means of powering the air ventilation system 108 or otherwise supporting a battery or external source, such as a solar panel located on the suit or faceplate, or through harnessing electricity from the wearer such as through their body heat.

In some embodiments, the air ventilation system 108 may be configured to be controlled by a computer processor directly connected to the suit or connected by a network connection to the suit, allowing a wearer to alter parameters of the suit through a control unit such as an arm-mounted computer or through a device such as a mobile phone. In some embodiments, the air ventilation system 108 may be controlled by a button 810, toggle, or other input located on the casing 808 which may control settings of the air ventilation system 108 or its activation or deactivation.

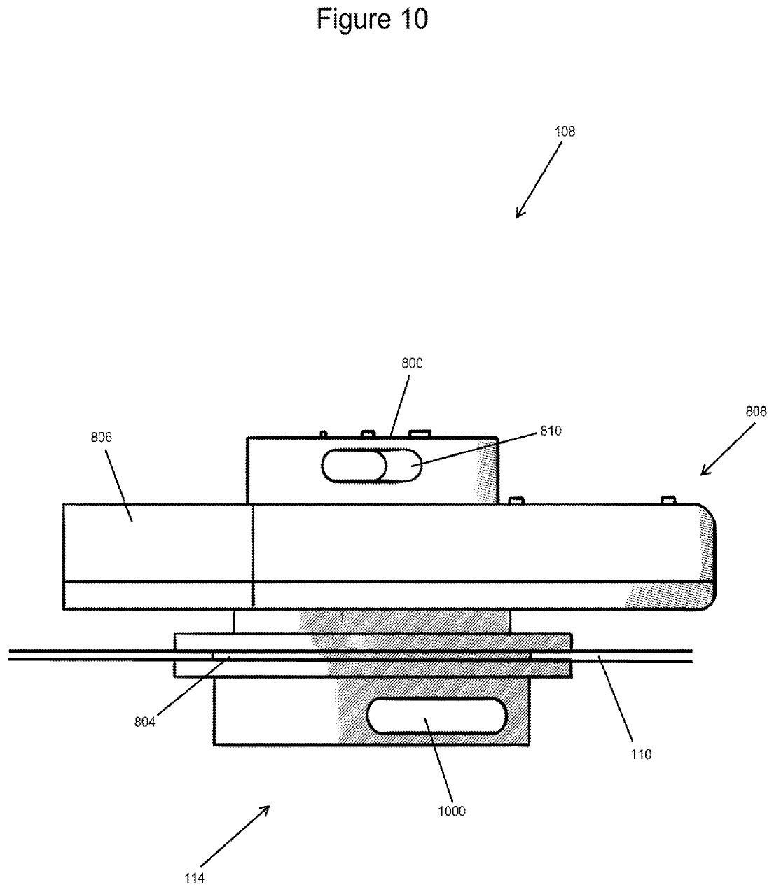

FIG. 10 is a side view of the air ventilation system of an integrated filtration system in accordance with an embodiment of the invention.

The air ventilation system 108, in some embodiments, is comprised of an air-flow assembly 800, a sanitation system 802, a power supply, a connection 804 to the faceplate 110, an exhaust port 806, and a casing 808. In some embodiments they may be separate components connected by a form of ducting, while in other embodiments they may be a singular component wherein air being pulled through the system by the air-flow assembly 800 is passed through the sanitation system 802 just before or just after it is taken into the fan. In some configurations, the air ventilation system 108 functions by operating a fan configured to push air out of the cavity 114 of the faceplate 110, and may be further comprised of a door, hatch, or similar blocking mechanism capable of ensuring that air cannot enter the faceplate 110 when the system is inoperative. Some configurations may involve the blocking mechanism being forced open by the increased air pressure that occurs in the cavity of the faceplate 110 when the wearer exhales, in which case the fan further pushes air out of the exhaust port 806 while the door is opened. In other embodiments, the door or blocking mechanism is closed when the fan is turned off to ensure air cannot leak out of the suit, and the door or blocking mechanism then opens when the fan is turned on.

In at least one embodiment, the connection 804 of the air ventilation system 108 extends at least partially into the cavity 114 of the faceplate 110 wherein are located one or more air intake apertures 1000 which connect to the fan assembly, which in turn leads to the blocking mechanism, and through that mechanism is an exhaust port 806 where air is forced by the fan out of the cavity 114 of the faceplate 110. The air ventilation system may have an integrated battery to power the fan and blocking mechanism. In some embodiments, the air ventilation system 108 may have one or more fan speed settings.

For example, in some embodiments, the air ventilation system 108 may be connected to an external air supply that may connect at the exhaust port 806 or at the connection 804 to the faceplate 110.

The sanitation system 802 of the air ventilation system 108 may be comprised of, in some embodiments, an ultraviolet-C(UVC) sanitation system wherein air leaving the system is passed through a UVC radiation source in order to sanitize it prior to leaving the suit's supply. In some embodiments, the air ventilation system 108 may further feature a filter to prevent unwanted particulates from leaving the cavity of the suit and to further ensure sanitation.

There are many mechanisms through which the air ventilation system 108 can be powered; some embodiments may rely on an internal battery, while others may connect to an external power source. Some embodiments of the invention may combine an internal battery and external source, or may incorporate an alternative means of powering the air ventilation system 108 or otherwise supporting a battery or external source, such as a solar panel located on the suit or faceplate, or through harnessing electricity from the wearer such as through their body heat.

In some embodiments, the air ventilation system 108 may be configured to be controlled by a computer processor directly connected to the suit or connected by a network connection to the suit, allowing a wearer to alter parameters of the suit through a control unit such as an arm-mounted computer or through a device such as a mobile phone. In some embodiments, the air ventilation system 108 may be controlled by a button 810, toggle, or other input located on the casing 808 which may control settings of the air ventilation system 108 or its activation or deactivation.

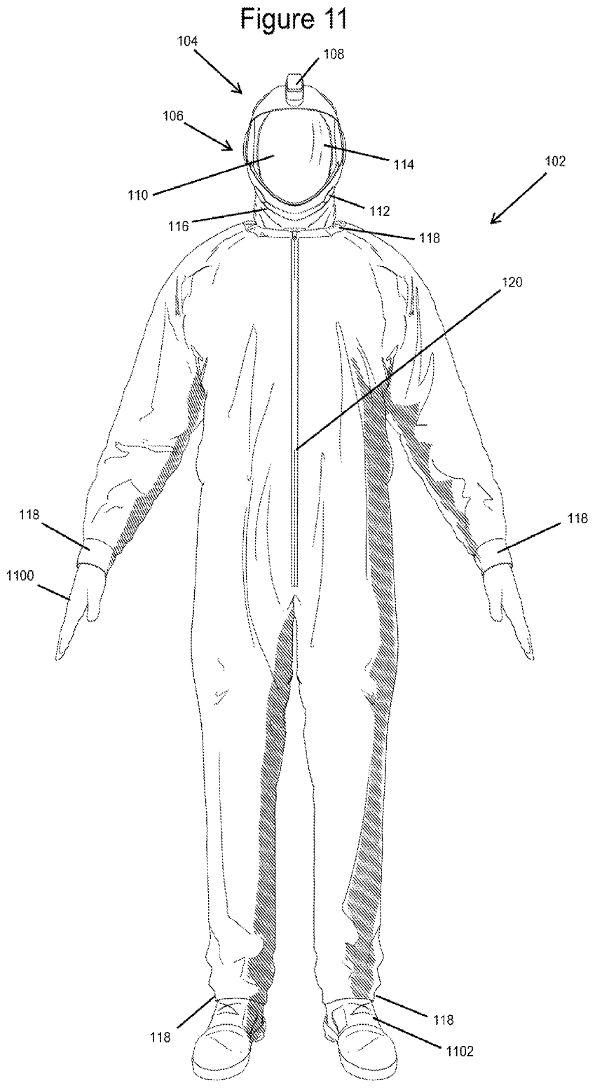

FIG. 11 is a front view of a personal protective suit with an integrated filtration system and integrated gloves and boots in accordance with an embodiment of the invention.

In some embodiments, the invention may be comprised of a personal protective suit 100 featuring three primary components; the body of the suit 102, head cover 104 featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket capable of receiving a filtering device wherein the pocket has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

In some embodiments, the suit 102 may be further comprised of integrated gloves 1100 and/or boots 1102, while in other embodiments the gloves 1100 and/or boots 1102 may be removably couplable, or held in place by the cuffs 118. Some embodiments may utilize a zipper, drawstring, or similar securing component in the cuffs 118 to couple the gloves 1100 and/or boots 1102 to the suit 102, while others may rely on friction or compression to hold the gloves 1100 and/or boots 1102 to the suit 102. In some embodiments, the suit has a zipper 120 integrated into the ventral side such that it is zipped from the front, while in other embodiments a zipper 120, zip-fastener, or other airtight securing means may be located on the dorsal or lateral sides of the suit.

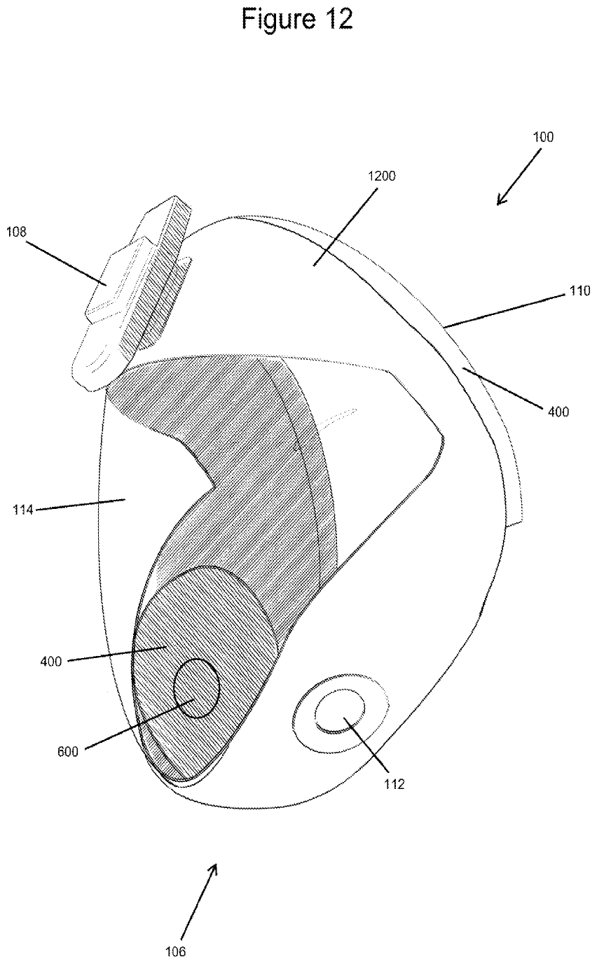

FIG. 12 is a perspective view of the mask component of a personal protective suit with an integrated filtration system in accordance with an embodiment of the invention.

In some embodiments, the invention may be comprised of a personal protective suit 100 featuring three primary components; the body of the suit 102, head cover featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter, and connection for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

The invention, in some embodiments, may also be comprised of cuffs 118 positioned at the wrists, neck, and ankles of the suit 102 wherein such cuffs 118 are configured to tightly grip the body of the wearer such that fluids are at least partially prevented from entering the suit 102.

Some embodiments of the invention are comprised of a faceplate that is further comprised of an at least partially translucent plate, a shell 1200 comprising at least a pocket, an air intake 112, a filter, and the air-ventilation system 108. In some embodiments, the invention is comprised of at least a faceplate assembly 106 that covers the face of the wearer wherein the faceplate assembly 106 is further comprised of a faceplate 110 and a pocket 400 capable of receiving a filtering device wherein the pocket 400 has at least one external intake port 112 and at least one internal intake port 600 capable of releasing air from the pocket 400 into the cavity 114; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system. In many embodiments, there will be two external intake ports 112 that correspondence with two internal intake ports 600 through which air is taken in, passed through the filter inside the pocket 400, and allowed into the cavity 114.

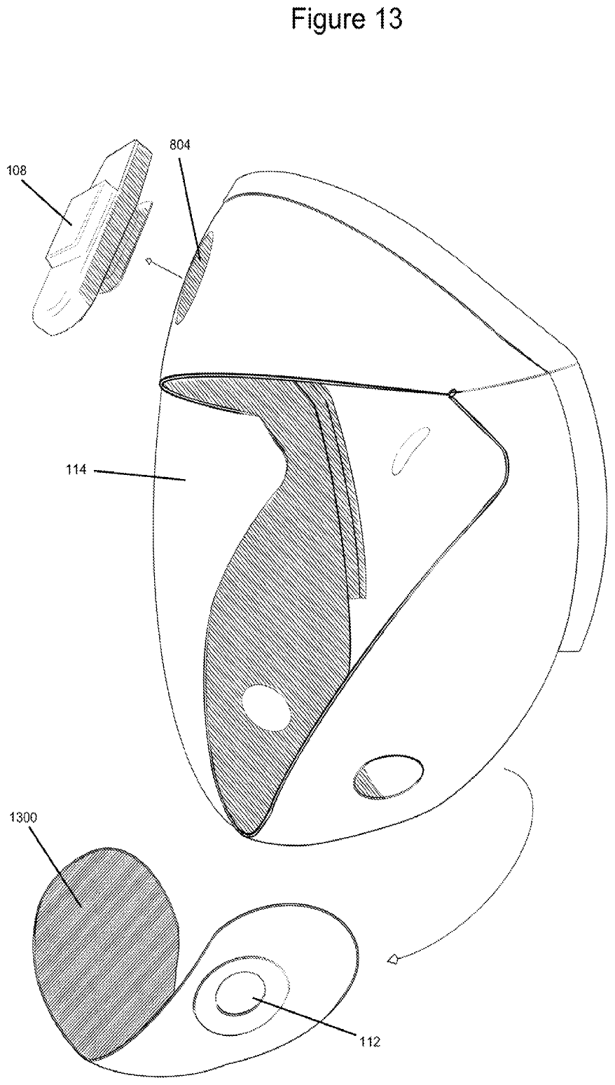

FIG. 13 is an exploded perspective view of the mask component of a personal protective suit with an integrated filtration system in accordance with an embodiment of the invention.

In some embodiments, the invention may be comprised of a personal protective suit 100 featuring three primary components; the body of the suit 102, head cover 104 featuring a faceplate assembly 106, and an air ventilation system 108. In some embodiments, additional components and functions may be added to the existing components. The body of the suit 102, in many embodiments, will cover the wearer from the ankles to wrists to neck and be comprised of a fluid-proof material capable of resisting at least water, but in many embodiments will be configured to be impermeable to other fluids. Some embodiments of the suit 102 may allow a fabric that is partially air permeable. The faceplate assembly 106, in many embodiments, is comprised of a faceplate 110, an intake port 112, filter 1300, and connection 804 for the air ventilation system 108 and couples with the suit 102 to form a cavity 114 of air over at least the wearer's nose and mouth. The air ventilation system 108, in many embodiments, is comprised of an air ventilation assembly 108 configured with a means to draw air from the cavity 114 of the faceplate assembly 106 and push it out, and may feature a sanitization system to sanitize the air before it exits the suit 102.

In some embodiments, the invention is comprised of a personal protective suit 100 for a wearer, comprising: a wearable garment comprised of a suit 102 configured to cover a wearer from the neck to the wrists and ankles in a fluid-tight material; a head cover 104 comprised of: a faceplate assembly 106 that covers the face of the wearer wherein the faceplate assembly 106 is further comprised of a faceplate 110 and a pocket 400 capable of receiving a filtering device wherein the pocket 400 has at least one intake port 112; a hood 116 that substantially covers the head of the wearer and may also cover their neck and shoulders and extends to the faceplate 110 and forms a fluid-tight seal with the faceplate 110 and forms a fluid-tight seal with the faceplate 110 to form a cavity 114 between the wearer's face and the faceplate 110; a filtering device configured to filter at least 95% of airborne particulates and configured to allow external air into the head cover 104 through the filtering device through the intake port 112 of the pocket 400 of the faceplate assembly 106; and an air ventilation system 108 configured to allow fluids to leave the cavity 114 when the air ventilation system 108 is activated without allowing fluids to enter regardless of whether the air ventilation system 108 is activated or deactivated and further comprised of a sanitization system configured to sanitize air before it leaves the air ventilation system.

Some embodiments of the invention are comprised of a faceplate assembly 106 that is further comprised of a faceplate 110 which may be at least partially translucent, a shell 1200 comprising at least a pocket 400, an air intake port 112, a filter 1300, and the air-ventilation system 108.

The pocket 400 system allows air to move through the air filter 1300 system when the wearer breathes, and the type of filter 1300 inserted into the filter system can be varied depending on the needs of the wearer. In some embodiments, the filter 1300 in the pocket may be an N95 filter, while in others it may be an N99, or other filtration mechanism as needed. In some embodiments, the pocket presses the filter 1300 against the air intake 112 of the pocket, ensuring that any air being brought into the suit is brought in only through the air intake 112 and thus must first pass through the filter 1300. Some embodiments may be configured such that the pocket is flexible and functions as a bladder, allowing the air pressure in the cavity 114 to be maintained as the wearer breaths.

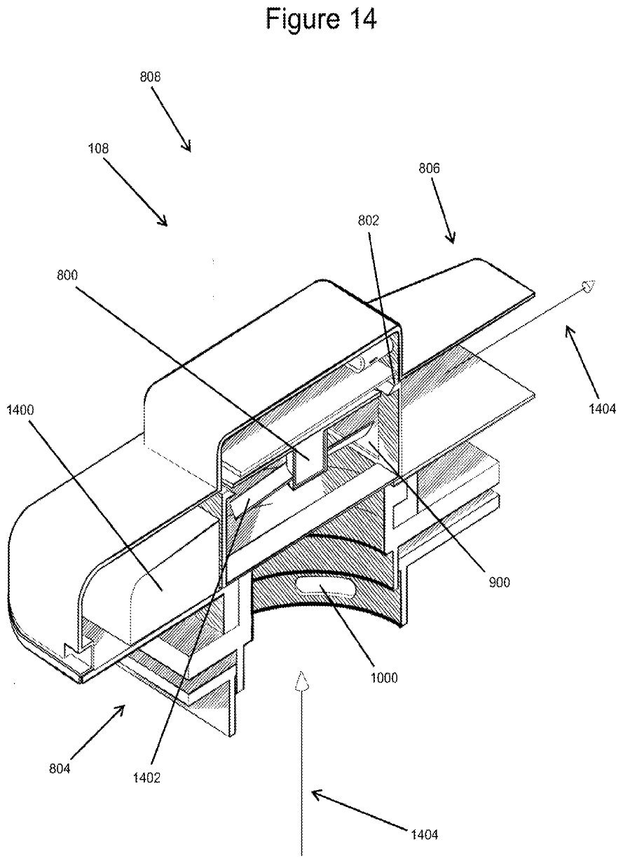

FIG. 14 is a perspective, cross-section view of the air ventilation system of an integrated filtration system in accordance with an embodiment of the invention.

The air ventilation system 108, in some embodiments, is comprised of an air-flow assembly 800, a sanitation system 802, a power supply 1400, a connection 804 to the faceplate, an exhaust port 806, and a casing 808. In some embodiments they may be separate components connected by a form of ducting, while in other embodiments they may be a singular component wherein air being pulled through the system by the air-flow assembly 800 is passed through the sanitation system 802 just before or just after it is taken into the fan 1402 of the air-flow assembly 800. In some configurations, the air ventilation system 108 functions by operating a fan 1402 configured to push air out of the cavity of the faceplate, and may be further comprised of a door 900, hatch, or similar blocking mechanism capable of ensuring that air cannot enter the faceplate when the system is inoperative. Some configurations may involve the door 900 or blocking mechanism being forced open by the increased air pressure that occurs in the cavity of the faceplate when the wearer exhales, in which case the fan 1402 further pushes air out of the exhaust port 806 while the door 900 is opened. In other embodiments, the door 900 or blocking mechanism is closed when the fan 1402 is turned off to ensure air cannot leak out of the suit, and the door 900 or blocking mechanism then opens when the fan 1402 is turned on. The arrows 1404 demonstrate the direction in which air flows through the air ventilation system 108.

In at least one embodiment, the connection 804 of the air ventilation system 108 extends at least partially into the cavity of the faceplate wherein are located one or more air intake apertures 1000 which connect to the fan 1402 assembly, which in turn leads to the door 900, and through that mechanism is an exhaust port 806 where air is forced by the fan 1402 out of the cavity of the faceplate. The air ventilation system 108 may have an integrated battery 1400 to power the fan 1402 and blocking mechanism. In some embodiments, the air ventilation system 108 may have one or more fan 1402 speed settings.

For example, in some embodiments, the air ventilation system 108 may be connected to an external air supply that may connect at the exhaust port 806 or at the connection 804 to the faceplate. In such embodiments, there may be a valve or similar mechanism to modulate the way the air leaves the suit.

The sanitation system 802 of the air ventilation system 108 may be comprised of, in some embodiments, an ultraviolet-C(UVC) sanitation system wherein air leaving the system is passed through a UVC radiation source in order to sanitize it prior to leaving the suit's supply. The sanitation system 802 may be located in whatever place is necessary but in some examples is located before the exhaust port 806, while in others it is positioned before the fan 1402. Some embodiments may locate the sanitation system 802 in other positions as well. In some embodiments, the air ventilation system 108 may further be comprised of a filter to prevent unwanted particulates from leaving the cavity of the suit and to further ensure sanitation. In some embodiments, the sanitation system 804 serves to ensure both that the air leaving the air ventilation system 108 is sanitized and to ensure that any air that does manage to return to the cavity is not contaminated.

There are many mechanisms through which the air ventilation system 108 can be powered; some embodiments may rely on an internal battery 1400, while others may connect to an external power source. Some embodiments of the invention may combine an internal battery 1400 and external source, or may incorporate an alternative means of powering the air ventilation system 108 or otherwise supporting a battery or external source, such as a solar panel located on the suit or faceplate, or through harnessing electricity from the wearer such as through their body heat.

In some embodiments, the air ventilation system 108 may be configured to be controlled by a computer processor directly connected to the suit or connected by a network connection to the suit, allowing a wearer to alter parameters of the suit through a control unit such as an arm-mounted computer or through a device such as a mobile phone. In some embodiments, the air ventilation system 108 may be controlled by a button or input located on the casing 808 which may control settings of the air ventilation system 108 or its activation or deactivation.

Although the invention has been described with reference to a particular example, it should be understood that it could be exemplified in many other ways. For instance, different styles of protective suits may be used, including suits that allow some leakage, and disposable suits. Also, rather than using an attached air ventilation system 108, external connected systems may be utilized.

Additional filters may be provided at the exhaust ports 806 to filter the breathing and ventilating air as it leaves the suit; this might be useful when the suit is to be worn in sterile environments such as clean rooms.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. Accordingly, the scope of the invention is not limited by the disclosure of these preferred and alternate embodiments. Instead, the invention should be determined by reference to the claims that follow.

What is claimed is:

1. A personal protective suit for a wearer, comprising:
a wearable garment comprised of a fluid-tight material;
a head cover that covers at least the nose and mouth of the wearer and forms a fluid-tight seal with the rest of the wearer's body, the head cover further comprising;
a filtering device configured to filter airborne particulates and configured to allow external air into the head cover through the filtering device; and
an air ventilation system configured to allow fluids to leave the head cover without allowing fluids to enter.

2. The personal protective suit of claim 1, wherein the wearable garment is shaped to cover the entire body of the wearer.

3. The personal protective suit of claim 1, wherein the wearable garment envelops at least the head of the wearer and creates a fluid-tight seal below the head of the wearer.

4. The personal protective suit of claim 3, wherein the fluid-tight seal of the head cover is located substantially along the perimeter of the human face.

5. The personal protective suit of claim 4, wherein the filtering device is further configured to filter out oils.

6. The personal protective suit of claim 5, wherein the filtering device is further configured to filter vapors and gases.

7. The personal protective suit of claim 2 wherein the head cover is removably couplable to the wearable garment.

8. The personal protective suit of claim 6, wherein the head cover is further comprised of a faceplate and the faceplate forms a cavity over a wearer's face with the boundary of said cavity beginning at the fluid-tight seal.

9. The personal protective suit of claim 8, wherein the air ventilation system is connected to the cavity formed by the head cover and is comprised of at least a fan assembly configured to direct air out of the cavity.

10. The personal protective suit of claim 9, wherein the filtering device is located on a bottom portion of the head cover and is further comprised of one or more air entry apertures.

11. The personal protective suit of claim 10, wherein the filtering device is further comprised of an air-permeable filter that covers the air entry apertures.

12. The personal protective suit of claim 11, wherein the air ventilation system is further configured to only permit fluids to leave the cavity of the face cover and not reenter through the air ventilation system.

13. The personal protective suit of claim 12, wherein the air ventilation system constantly pulls air from the cavity formed by the faceplate and expels it outside the suit.

14. The personal protective suit of claim 13, wherein the air ventilation system is configured with at least two settings which determine the rate at which it pulls air from the cavity.

15. The personal protective suit of claim 12, wherein the air ventilation system is further comprised of a light-based sanitation device positioned and configured to irradiate air before it leaves the air ventilation system.

16. The personal protective suit of claim 15, wherein the light-based sanitation device of the air ventilation system utilized ultraviolet light to purify the air.

17. The personal protective suit of claim 13, wherein the fluid-tight seal of the head cover can be adjusted by the wearer.

18. The personal protective suit of claim 17 wherein the filtering device of the head cover is removable when the suit is not being worn.

19. A personal protective suit for a wearer, comprising:
a wearable garment comprised of a fluid-tight material;
a head cover comprised of:
a faceplate that covers at least the nose and mouth of the wearer and forms a cavity over the area covered with a fluid-tight seal to the outside of the cavity;
a filtering device configured to filter at least 95% of airborne particulates and configured to allow external air into the head cover through the filtering device; and
an air ventilation system configured to allow fluids to leave the cavity without allowing fluids to enter and further comprised of a sanitization system configured to sanitize air before it leaves the air ventilation system.

20. A personal protective suit for a wearer, comprising:
a wearable garment comprised of a suit configured to cover a wearer from the neck to the wrists and ankles in a fluid-tight material;
a head cover comprised of:
a faceplate assembly that covers the face of the wearer wherein the faceplate assembly is further comprised of a faceplate and a closeable bladder comprising a pocket capable of receiving a filtering device wherein the bladder has at least one aperture;
a hood that substantially covers the head of the wearer up to the faceplate and forms a fluid-tight seal with the faceplate to form a cavity between the wearer's face and the faceplate;
a filtering device configured to filter at least 95% of airborne particulates and configured to allow external air into the head cover through the filtering device through the aperture of the pocket of the faceplate assembly; and
an air ventilation system configured to allow fluids to leave the cavity when the air ventilation system is activated without allowing fluids to enter regardless of whether the air ventilation system is activated or deactivated and further comprised of a sanitization system configured to sanitize air before it leaves the air ventilation system.
